(19) United States
(12) Patent Application Publication
(10) Pub. No.: US 2020/0237496 A1
(43) Pub. Date: Jul. 30, 2020

(54) ENDOVASCULAR REPLACEMENT OF AORTIC VALVE, AORTIC ROOT, AND ASCENDING AORTA

(71) Applicant: KHOYNEZHAD, ALI, MANHATTAN BEACH, CA (US)

(72) Inventor: Ali Khoynezhad, Manhattan Beach, CA (US)

(21) Appl. No.: 16/634,073

(22) PCT Filed: Jul. 25, 2018
(86) PCT No.: PCT/US2018/043773

§ 371 Date: Jan. 24, 2020

Related U.S. Application Data
(60) Provisional application No. 62/672,958, filed on May 17, 2018.
(60) Provisional application No. 62/536,617, filed on Jul. 25, 2017.

(51) Int. Cl. A61F 2/07; A61F 2/95; A61F 2/24

(52) U.S. Cl. CPC A61F 2/07; A61F 2002/061; A61F 2/2418; A61F 2/95

(57) ABSTRACT

The present disclosure relates generally to methods and apparatus for endovascular replacement of the aortic valve, aortic root, and ascending aorta. The present subject matter includes various embodiments of methods and apparatus for connecting the coronary arteries to an endovascular assembly including an aortic valve device (AVD), an aortic root graft, and an ascending aortic stent graft. The present subject matter provides for replacement of the aortic valve, aortic root, and ascending aorta without requiring open-heart surgery. It provides different options for connections to the coronary arteries and for assembling the aortic valve device and the aortic root graft.

### RELATED APPLICATIONS

This application claims the benefit of priority under 35 U.S.C. § 119(e) of U.S. Provisional Patent Application Ser. No. 62/672,958, filed on May 17, 2018, entitled “Endovascular Replacement of the Aortic Valve, Aortic Root, and Ascending Aorta Method and Apparatus” and U.S. Provisional Patent Application Ser. No. 62/536,617, filed on Jul. 25, 2017, entitled “Endovascular Replacement of the Aortic Valve, Aortic Root, and Ascending Aorta Method and Apparatus (Endo-Bentall procedure),” both applications of which are incorporated by reference in their entirety.

### TECHNICAL FIELD

Embodiments described herein relate generally to methods and apparatus for endovascular replacement of the aortic valve, aortic root, and ascending aorta.

### BACKGROUND

Any organism having a heart and ascending aorta present varying degrees and types of aortic and cardiac disease, which can require treatment or replacement of the aortic valve, aortic root, and ascending aorta. Open-heart surgical methods and apparatus have been developed which allow for repair and replacement of the aorta and heart anatomy in humans and animals; however, such surgical methods are invasive and result in higher surgical complication risk and prolonged recovery time, especially in elderly and high-risk and re-do operative setting.

Some subjects are eligible for replacement or repair of blood vessels and cardiac anatomy using endovascular techniques, such as endovascular heart valve replacement. The complexity of such replacement and repair increases substantially should multiple endovascular procedures be required to treat the patient. Patients who require replacement of the aortic valve, aortic root, and ascending aorta were heretofore unable to obtain such replacements using a single endovascular device and procedure and therefore likely to be subjected to open-heart surgery. Furthermore, device solutions employing multiple components risk endoleak. Poorly fitting designs with multiple components can also cause narrowing or hourglass shaping of a lumen of the aortic root or the ascending aorta and can cause a local stenosis or narrowing in the proximal aorta. Such narrowing or hourglass shaping of the inner lumen of the aortic root or the ascending aorta can cause gradients and pressure difference within the assembly and increase afterload and workload for the heart.

There is a need in the art for methods and apparatus that provide endovascular replacement of the aortic valve, aortic root, and ascending aorta of humans or other living animals using a simpler procedure, without the need of opening the sternum and open-heart surgery. There is also a need for methods and apparatus which do not cause endoleak, and gradients and pressure issues that can cause afterload and workload for a patient's heart.

### SUMMARY

The present disclosure relates generally to methods (the Endo-Bentall procedure) and apparatus (Endo-Bentall device) for endovascular replacement of the aortic valve, aortic root, and ascending aorta. The present subject matter includes various embodiments of methods and apparatus for connecting the coronary arteries to an endovascular assembly including a commercially available transcatheter aortic valve or suture-less aortic valve or surgical valves (all combined in the following referred as aortic valve device or AVD), an aortic root graft, and an ascending aortic stent graft. The present subject matter provides for replacement of the aortic valve, aortic root, and ascending aorta without requiring open-heart surgery or heart-lung machine. It provides different options for connections to the coronary arteries and for assembling the transcatheter aortic valve and the aortic root graft.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

The present disclosure is illustrated by way of example and not limitation in the figures of the accompanying drawings, in which like references indicate similar elements and in which:

FIG. 1 is an anatomical drawing of an aneurysm of the ascending aorta, the aortic arch, the aortic root, and the right and left coronary arteries.

FIGS. 2A and 2B demonstrate the convergence of an Endo-Bentall device with a commercially available AVD: In this version of embodiment, plurality of hooks and other connectors (2A) at the proximal end of the Endo-Bentall to allow the assembly with the AVD according to one embodiment of the present subject matter.

FIGS. 3A and 3B demonstrate the convergence of an Endo-Bentall device with a commercially available AVD: In this version of embodiment, a snare in the proximal cloth (3A) allows the “skirted” assembly of the Endo-Bentall device with the AVD according to one embodiment of the present subject matter. The snare is tightened and subsequently knotted to complete this assembly.

FIG. 4 is a drawing illustrating endovascular delivery (in this case trans-femoral route) of one example of an Endo-Bentall device in a collapsed state to enable delivery over a wire and as a unified device, according to one embodiment of the present subject matter.

FIG. 5 is a drawing illustrating endovascular delivery of one example of the present stent graft device in a partially deployed state, with AVD being deployed in proximal “landing zone” (aortic valve annulus), and fenestrations for coronary stents exposed in the aortic root graft, according to one embodiment of the present subject matter.

FIG. 6 is a drawing demonstrating one example of an Endo-Bentall device including fenestrations connected to the right and left coronary arteries using commercially available coronary stent grafts, according to one embodiment of the present subject matter.

FIG. 7 is a drawing demonstrating one example of an Endo-Bentall device including an internal conduit (within aortic lumen) connected to right and left stent graft branches configured to be connected to the right and left coronary arteries, respectively, according to one embodiment of the present subject matter. The two internal branches are bridged to the right and left coronary arteries using commercially available coronary stent grafts.

FIG. 8 is a drawing demonstrating one example of an Endo-Bentall device including two separate conduits each having an orifice to the aortic lumen, and configured to be connected to the right and left coronary arteries, respectively, according to one embodiment of the present subject matter. In this embodiment, the conduits connect to stent grafts that are either immediately externalized (compared to the ascending stent graft) 826, or connect to an external stent graft after a short segment of an internal conduit 824.

FIG. 9 is a drawing demonstrating one example of an aortic stent graft including a shorter internal conduit connected to right and left stent graft branches configured to be connected to the right and left coronary arteries, respectively, according to one embodiment of the present subject matter.

FIG. 10 is a drawing demonstrating a base of an Endo-Bentall device, according to various embodiments of the present subject matter.

FIG. 11 is a drawing demonstrating an Endo-Bentall device formed as a spiral stent (inside or outside of the polyester or PTFE graft) with longitudinal bar and a base with a plurality of fenestrations, according to various embodiments of the present subject matter.

FIG. 12 is a drawing demonstrating detail of the fenestrations and connections with an AVD according to various embodiments of the present subject matter.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

The present disclosure relates generally to methods (the Endo-Bentall procedure) and apparatus (Endo-Bentall device) for endovascular replacement of the aortic valve, aortic root, and ascending aorta. The present subject matter includes various embodiments of methods and apparatus for connecting the coronary arteries to an endovascular assembly including an AVD, an aortic root graft, and an ascending aortic stent graft. The present subject matter provides for replacement of the aortic valve, aortic root, and ascending aorta without requiring open-heart surgery or heart-lung machine. It provides different options for connections to the coronary arteries and for assembling the AVD and the aortic root graft.

The present subject matter addresses several aortic root pathologies including, but not limited to, aortic aneurysm, acute and chronic aortic dissection, traumatic aortic transection and iatrogenic aortic injury, aortic pseudo-aneurysm, intramural hematoma, and penetrating ulcer of the aortic root and the ascending aorta. Since around 70% of aortic pathologies are found in the aortic root, it will impact a large population who heretofore had a risky open-heart surgery as only alternative. The present subject matter allows for endovascular exclusion and implantation of a device that replaces the 1) aortic valve, 2) aortic root, 3) connections to the coronary arteries and 4) the ascending aorta and/or the aortic arch.

In various embodiments, a unitary device is configured to provide the ascending stent graft, aortic root graft, and AVD device. Embodiments using this singular body design remove the risk of endoleak compared to designs involving two or more components for aortic coverage.

Furthermore, such unitary device embodiments are sized to prevent narrowing or hourglass shaping of a lumen of the aortic root or the ascending aorta, when compared to designs which have two or more components connecting together and which can cause a local stenosis or narrowing in the proximal aorta. Such narrowing or hourglass shaping of the inner lumen of the aortic root or the ascending aorta would likely cause gradients and pressure difference within the assembly and increase afterload and workload for the heart.

Additionally, the AVD used in unitary device embodiments could be most off-the-shelf transcatheter valves, which are ubiquitous and already FDA approved, and deployed per instructions for use (IFU). This is clearly an advantage to other embodiments involving newly patented valve designs that need to be tested for in-vitro and in-vivo before the use in any endo-Bentall procedure and pre-assembled within the aortic graft. The AVD in such embodiments may be ballon expandable or self-expanding, while other embodiments have only ballon-expandable stent grafts.

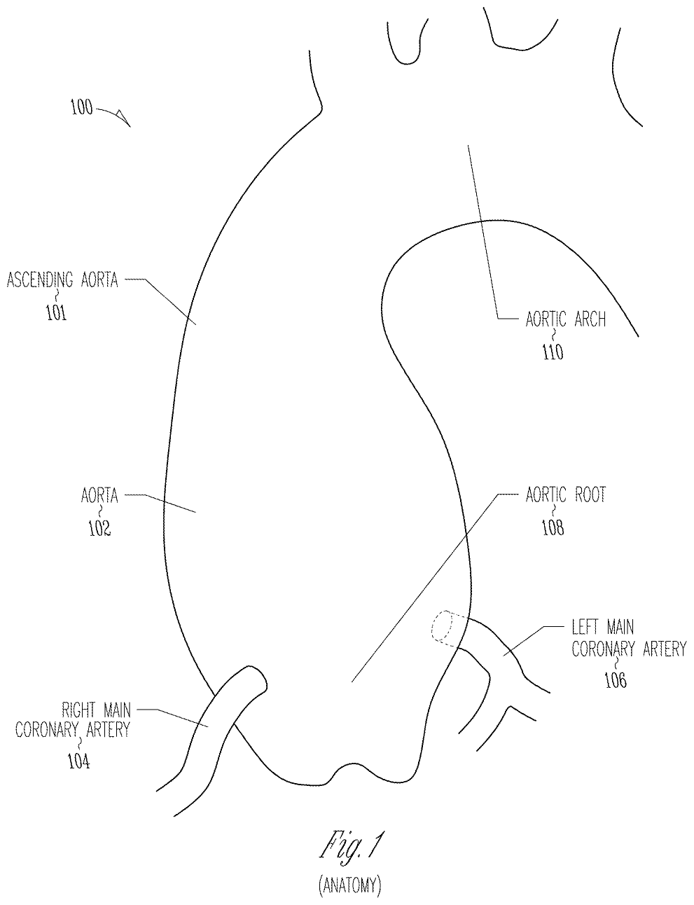

FIG. 1 is an anatomical drawing of the aortic arch, an aneurysm of the ascending aorta, and the aortic root, with typical configuration of the right and left coronary arteries. It shows anatomy 100 including an aorta 102 and an ascending aorta 101 in fluid communication with an aortic arch 110. The aorta 102 includes an aortic root 108, which is in fluid communication with the right coronary artery 104 and the left coronary artery 106.

The present subject matter enables an Endo-Bentall device to be connected to a commercially available AVD or a specially designed AVD. In various embodiments, the Endo-Bentall device is assembled with the AVD connected and ready for implantation in pre-assembled commercially available option. In various embodiments, the Endo-Bentall device is connected to the AVD in the operating room just prior to the Endo-Bentall procedure. In various embodiments, the Endo-Bentall device is connected to the AVD in-situ to allow for independent implantation of the Endo-Bentall device and of the AVD. Such connections may be employed by the following approaches, including but not limited to, suturing or snaring down, hooks, mechanical connectors or sliding and/or binding (magnetic, chemical and/or nano-technology enabled) surfaces. In various applications, the connections are made in the hybrid operating room, catheterization laboratory or a surgical operating room. Other connection means and methods may be employed without departing from the scope of the present subject matter.

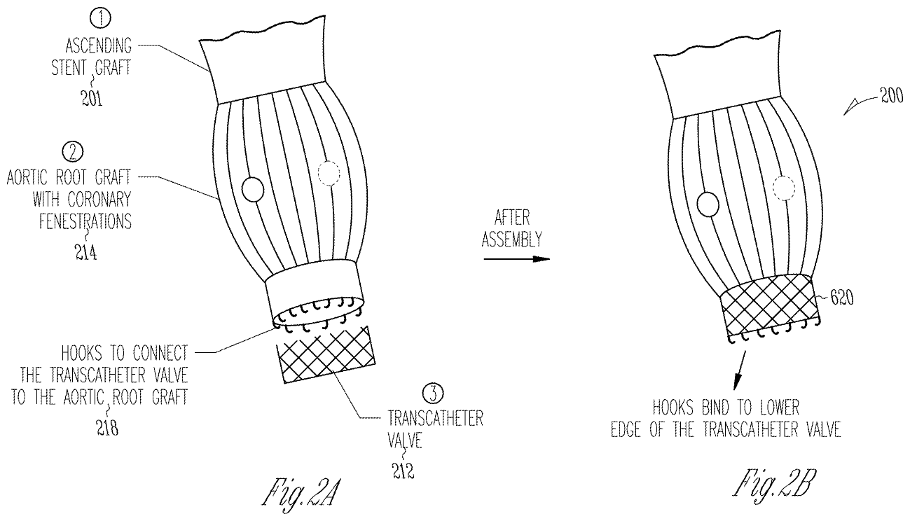

FIGS. 2A and 2B demonstrate two embodiments of how the Endo-Bentall device would converge with a commercially available AVD: the use of a plurality of hooks and mechanical connectors (2A) to connect an aortic root graft to an AVD, according to one embodiment of the present subject matter. In various embodiments, aortic root graft 214 comes pre-assembled with ascending stent graft 201. Aortic root graft 214 is fitted proximally with hooks 218 that can bind to the lower edge of the AVD or transcatheter aortic valve 212. Another embodiment (not shown) allows the AVD 212 to be independently sutured using a running polypropylene suture into the proximal end of the Endo-Bentall device (combined aortic root graft 214 and ascending stent graft 201). This connection facilitates the individual deployment of portions of device assembly 200, and their subsequent assembly in-situ. Those of skill in the art upon reading and understanding the present disclosure will appreciate that other embodiments are possible without departing from the scope of the present subject matter.

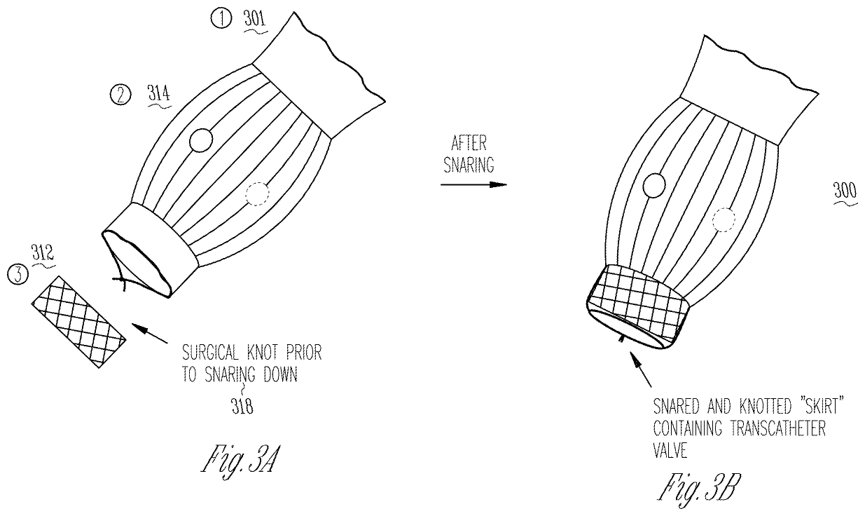

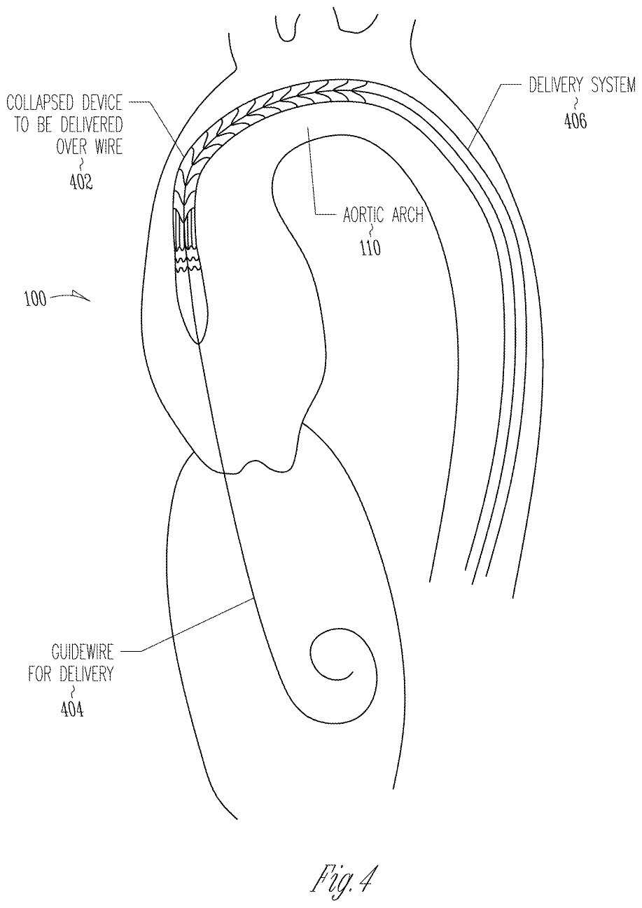

FIGS. 3A and 3B demonstrate the use of a snare and knotted skirt to contain an AVD to connect an aortic root graft to a transcatheter valve, according to one embodiment of the present subject matter. In various embodiments, aortic root graft 314 comes pre-assembled with ascending stent graft 301. The proximal segment of the aortic root graft 314 is fitted with a suture or tape that may be snared after insertion of the AVD 312. The suture or tape is snared down and knotted, enabling the secure assembly of the AVD 312. It allows AVD 312 to be independently sutured into position and later combined with aortic root graft 314 and ascending stent graft 301. This connection facilitates the individual deployment of portions of device assembly 300, and their subsequent assembly in-situ. Those of skill in the art upon reading and understanding the present disclosure will appreciate that other embodiments are possible without departing from the scope of the present subject matter.

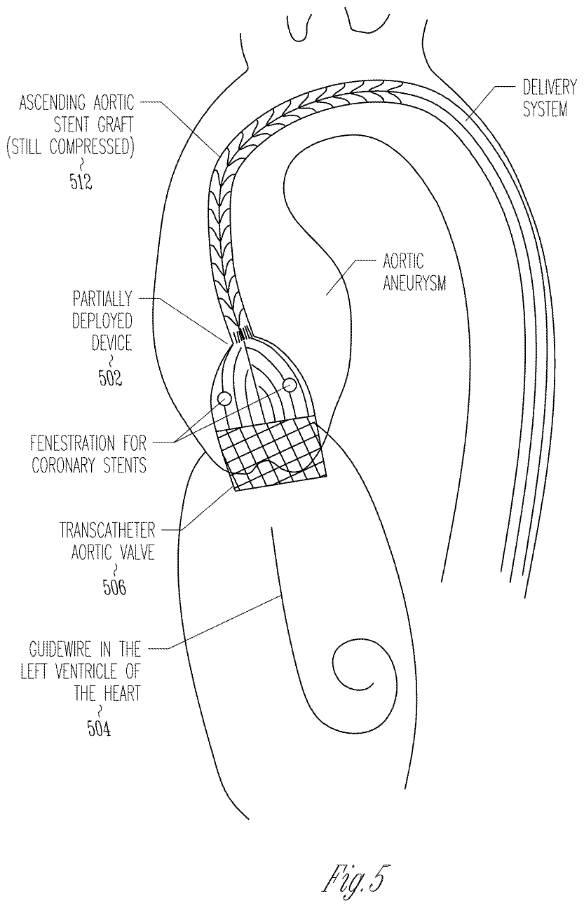

FIG. 4 is a drawing illustrating endovascular delivery of one example of the present stent graft device in a collapsed state to enable delivery over a wire, according to one embodiment of the present subject matter. In this example, the stent graft 402 is delivered over the wire 404, placed in the left ventricle of the heart, in a collapsed or reduced state to facilitate endovascular passage through the portions of the vasculature. In various embodiments the device is delivered through femoral arteries. In various embodiments it is delivered through alternative access routes including, but not limited to, trans-apical (apex of the heart), trans-subclavian, trans-carotid, trans-axillary, and trans-aortic (direct ascending aortic access).

In various embodiments, the device includes: 1) a cloth (made of polyester, Dacron or PTFE) that will bond to the AVD; 2) a PTFE, Dacron or polyester based aortic root segment with orifices accommodating 3) two fenestrations (simple wire-reinforced orifices with radiopaque markings) or two branch stent grafts (internal running smaller circular stent grafts that allows connection the orifices of the left and right coronary artery); and 4) an ascending aorta segment made of a nitinol or titanium or similar metal reinforced polyester or PTFE stent graft for distal “landing zone” (distal ascending aortic or aortic arch seal of the Endo-Bentall). Therefore, various embodiments of the present subject matter are sealed proximally at the aortic valve and distally at the distal ascending aorta or the aortic arch. The ascending aortic graft (301) and aortic root graft (314) may have incorporated bars (or wires) or stents to increase the radial force. This stents/bars may be longitudinal (shown in 314) round (shown in 301), spiral (shown in FIG. 1), continuous (shown in FIG. 1), interrupted (shown in 301) or other constellation. This radial force allows for apposition of the coronary fenestrations (214) to the left and right coronary arteries in patients, where the aortic root is normal in size, such as type A dissection or ascending aneurysm without aortic root involvement. In such cases, a coronary stent graft (704) would not be needed.

Before implantation, the selected AVD may be opened sterilely along according to the present subject matter and connected by means of suturing or snaring down, hooks, mechanical connectors or sliding and/or binding (magnetic, chemical and/or nano-technology enabled) surfaces in the hybrid operating room, catheterization laboratory or a surgical operating room. Various embodiments include a device that already has an AVD assembled, and such embodiments may be connected in the sterile facility by a device manufacturing company and inserted into the delivery system and ready for implantation. The sizes of the AVD is based on CT measurements of the aortic valve annulus. The aortic root and ascending aortic portions (nos. 2 and 4 as listed in previous paragraph) are sized based on size of “non-diseased” distal aorta. Therefore, in various embodiments, the approach may be individualized based on subject's anatomy and delivered via a covered delivery system as demonstrated in the attached drawings.

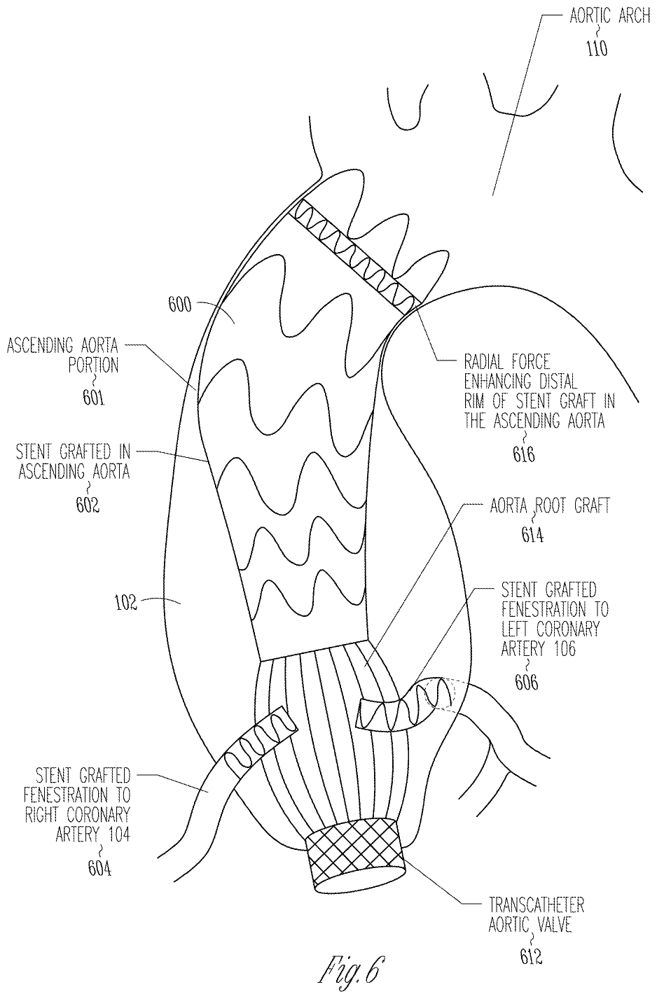

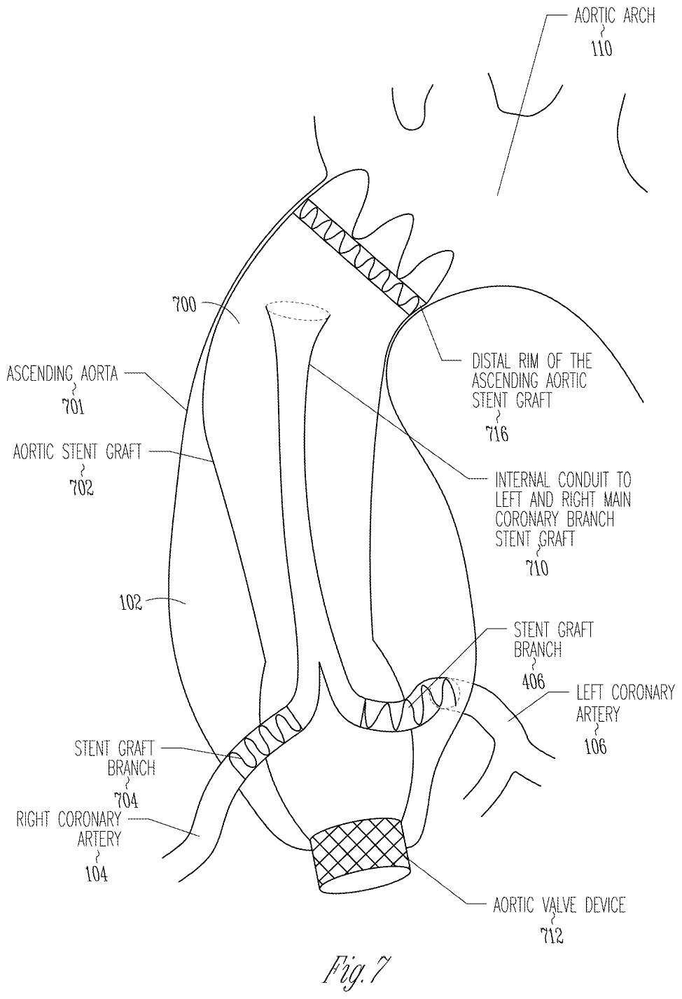

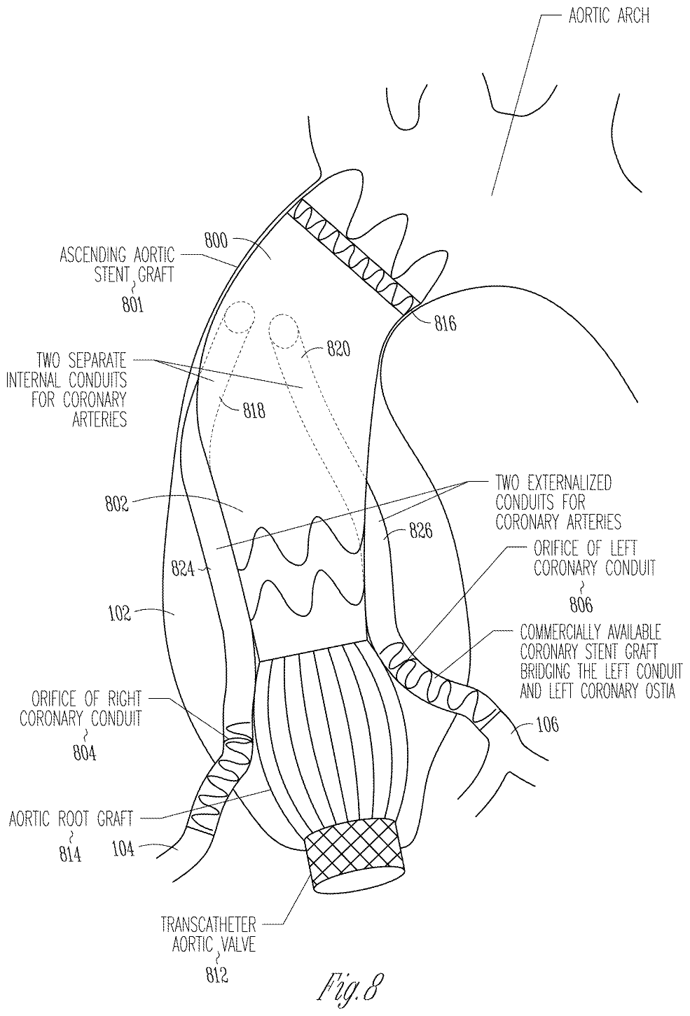

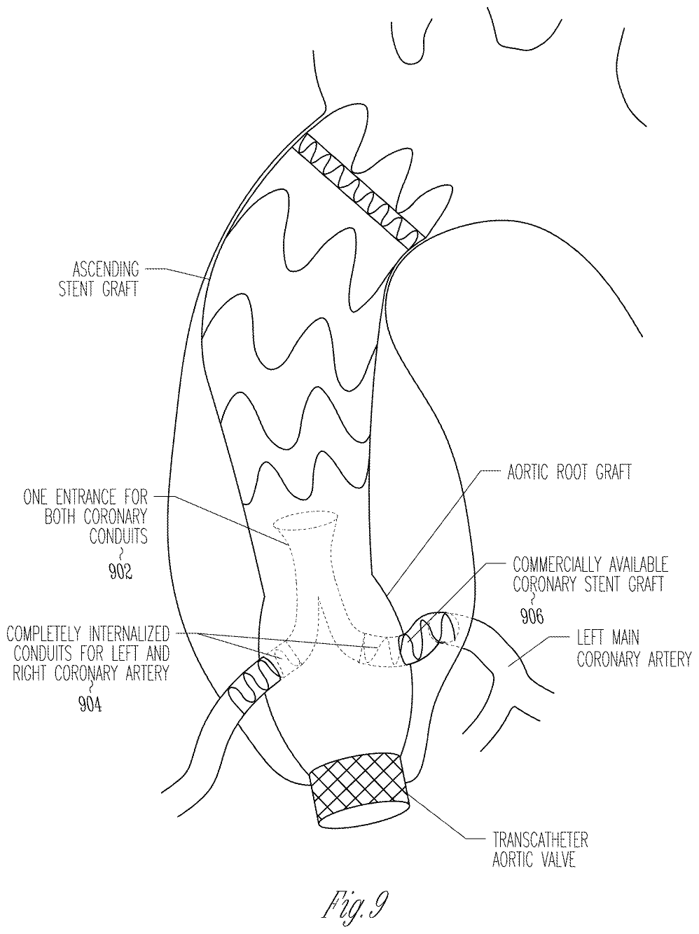

The orifices for the left and right coronary arteries 104-106 in various embodiments of the aortic root portion are typically 4-8 mm in diameter and circular. In embodiments where there are internal conduits and branches, (such as in FIG. 7, instead of fenestrations, such as in FIG. 6) to the coronary arteries, they are nitinol, titanium or similar metal reinforcing the PTFE or Dacron or polyester and run internally (inside ascending stent graft lumen), externally (outside of stent graft lumen but within the aorta) or a combination of both in the aortic root portion and are in continuation with the stent graft in the ascending aorta. Furthermore, the distal end may be in the aortic root (FIG. 9), proximal or distal ascending aorta (FIGS. 7, 8). The distal aortic component can be unified in one reverse conical shape (see FIG. 7) or two conical, oval or round shapes (FIG. 8). The purpose of this shape is to allow for easy transfemoral cannulation of these lumina with a guidewire and subsequent placement of commercially available stent grafts to bridge the gap between the coronary artery ostia and the proximal end of the coronary branch stent graft or fenestration (depending on which one of the various embodiments is chosen). The location of the coronary artery fenestration (FIG. 5) or branch stent grafts (604, 606) may be adjusted according to the patient's coronary anatomy constellation. While ca. 90% of patient will have similar spatial distance/angulation (FIG. 5), for patients with aberrant coronary anatomy, the location/constellation of fenestrations/coronary stent grafts can be custom-made. Alternatively, the fenestrations may be performed on the operating table and prior to implantation using a cutting or burning device (such as eye cautery) to produce the proper fenestration (not shown). Alternatively, for patients with one anatomic or functional coronary artery, variations exist in which where only one fenestration or branch stent graft, or alternatively a stent graft plug may be inserted to obstruct the orifice of this coronary connection (not shown). The plug looks like a stent graft with flush obstruction of entire lumen with cloth, similar to plugs used in commercially available aorto-uni-iliac stent grafts for abdominal aortic aneurysms. Those of skill in the art upon reading and understanding the present disclosure will appreciate that other embodiments are possible without departing from the scope of the present subject matter.

In various embodiments, the device will have reinforcement with circumferential nitinol, titanium or similar metals for stronger radial force at the aortic valve annulus (FIG. 712) aortic root graft (314) and distal ascending aorta (FIG. 716). Those of skill in the art upon reading and understanding the present disclosure will appreciate that other materials may be used.

In the operating room and after proper sizing, the Endo-Bentall device is crimbeled into the delivery sheath after the AVD is incorporated in the proximal section. FIG. 5 is a drawing illustrating endovascular delivery of one example of the present Endo-Bentall device 502 in a partially deployed state having sealed the proximal “landing zone” with the AVD, and exposed fenestration for coronary stents, according to one embodiment of the present subject matter. In one example of endovascular delivery a partially deployed device with fenestration for coronary stents is disposed to treat an aortic aneurysm using a guidewire 504 inserted into the aorta and left ventricle of the heart. Under fluoroscopic guidance the AVD segment of the delivery system 506 is positioned in the native aortic annulus and deployed, followed by aortic root graft, and subsequently the ascending aortic stent graft 512.

FIG. 6 is a drawing demonstrating one example of an aortic stent graft including stent grafted fenestrations to the right and left coronary arteries after completed deployment, according to one embodiment of the present subject matter. It shows an endovascular device assembly (Endo-Bentall device) 600 disposed in aorta 102 such that stent graft 602 includes an ascending aorta portion 601 in the ascending aorta 101 with a radial force enhancing distal rim 616 of the stent graft in the ascending aorta 101. The ascending stent graft 602 is connected to an aortic root graft 614 having stent grafted fenestrations 604 and 606 configured to be connected to the right coronary artery 104 and left coronary artery 106 respectively. The aortic root graft 614 is configured to connect with an AVD 612. As demonstrated in this specification, the device assembly 600 may allow for different connections between aortic root graft 614 and AVD 612, including, but not limited to those demonstrated by FIGS. 2A, 2B, 3A, and 3B.

FIG. 7 is a drawing demonstrating one example of the Endo-Bentall device including an internal conduit—with an oversized oval or round internal orifice—connected to right and left stent graft branches configured to be connected to the right and left coronary arteries, respectively, according to one embodiment of the present subject matter. It shows an endovascular device assembly 700 disposed in aorta 102 such that an aortic stent graft 702 placed in the ascending aorta 701 with a distal “landing zone” 716 of the stent graft in the ascending aorta 101 or the aortic arch. In patients with aortic arch pathologies such as aneurysm, the distal segment may be placed in the aortic arch or the descending thoracic aorta. This distal extension can be accomplished with manufactured branched aortic arch stent graft devices and is not an innovation claimed by this disclosure. The stent graft 702 includes an internal conduit 710 connected to a right stent graft branch 704 and a left stent graft branch 706, which are configured to be connected to the right coronary artery 104 and the left coronary artery 106, respectively. The aortic stent graft 702 is connected to an AVD 412. The Endo-Bentall device 700 may allow for different connections between aortic stent graft 702 and AVD 712, including, but not limited to those demonstrated by FIGS. 2A, 2B, 3A, and 3B. In various embodiments, the internal conduit 710 or the coronary fenestration 604/606 is reinforced with radial stents to avoid compressions.

FIG. 8 is a drawing demonstrating one example of an aortic stent graft including separate conduits each having an internal orifice configured to be connected to the right and left coronary arteries, respectively, according to one embodiment of the present subject matter. It shows an endovascular device assembly 800 disposed in aorta 102 such that an aortic stent graft 802 includes an ascending aorta portion 501 in the ascending aorta 101 with a distal rim 816 of the stent graft in the ascending aorta 101 with enforced rows of stent graft to increase the radial force at distal “landing zone” 816. The Endo-Bentall device 800 includes dual internal conduits 818 and 820 connected to corresponding plural external conduits 824 and 826 terminating in a right orifice 804 and left orifice 806, respectively, which are configured to be connected to the right coronary artery 104 and left coronary artery 106, respectively. In this embodiment, the conduits connect to stent grafts that are either immediately externalized (compared to the ascending stent graft) 826, or connect to an external stent graft after a short segment of an internal conduit 824. This spectrum of conduit choices will allow for individual device selection according to subject's aortic anatomy. In various embodiments, a commercially available coronary stent graft may be used to bridge the external conduits (824,826). Other stent grafts may be used without departing from the scope of the present subject matter.

The ascending stent graft 802 is connected to an aortic root graft 814. The aortic root graft 814 is configured to connect with an AVD 812. As demonstrated in this specification, the device assembly (Endo-Bentall device) 800 may allow for different connections between aortic root graft 814 and AVD 812, including, but not limited to those demonstrated by FIGS. 2A, 2B, 3A, and 3B.

FIG. 9 is a drawing demonstrating another embodiment of the deployed Endo-Bentall device including an internal conduit connected to right and left stent graft branches configured to be connected to the right and left coronary arteries, respectively, according to one embodiment of the present subject matter. In this embodiment, the single internal conduit 902 is short and starts in the proximal ascending aorta or distal aortic root. After a short common trunk, the internal conduit divides into left and right internal conduits 904 that end at the external surface of the aortic root graft. In this embodiment, the entire coronary conduit is internal 904 and within the Endo-Bentall device lumen. In various embodiments, commercially available coronary stent grafts may be used to bridge the internal conduits. In various embodiments, the internal orifice of the single internal conduit 902 can be oval or round. In various embodiments, the cross section of the internal left and right conduits can be oval or round. In various embodiments, the cross section of the coronary stent grafts 906 can be oval or round.

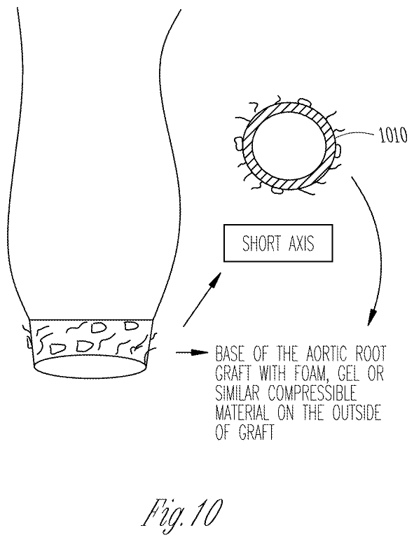

FIG. 10 is a drawing demonstrating a base of Endo-Bentall device, according to various embodiments of the present subject matter. The external base 1010 of the aortic root graft can be made of different materials to eliminate or reduce paravalvular leaks. These materials would be adherent to the proximal “landing zone” of the aortic root graft. In various embodiments, the base 1010 includes foam, gel or other compressible material or a combination of these materials attached to the external base of the aortic root graft to eliminate or reduce paravalvular leak. In various embodiments, the base 1010 includes a radially stretchable polyester or pTFE to allow for expansion of the commercially available AVD to the aortic valve annulus during the cardiac cycle. Other materials are possible without departing from the scope of the present subject matter. It is understood that the base demonstrated in FIG. 10 can be used with the other aortic root graft embodiments described herein in combination.

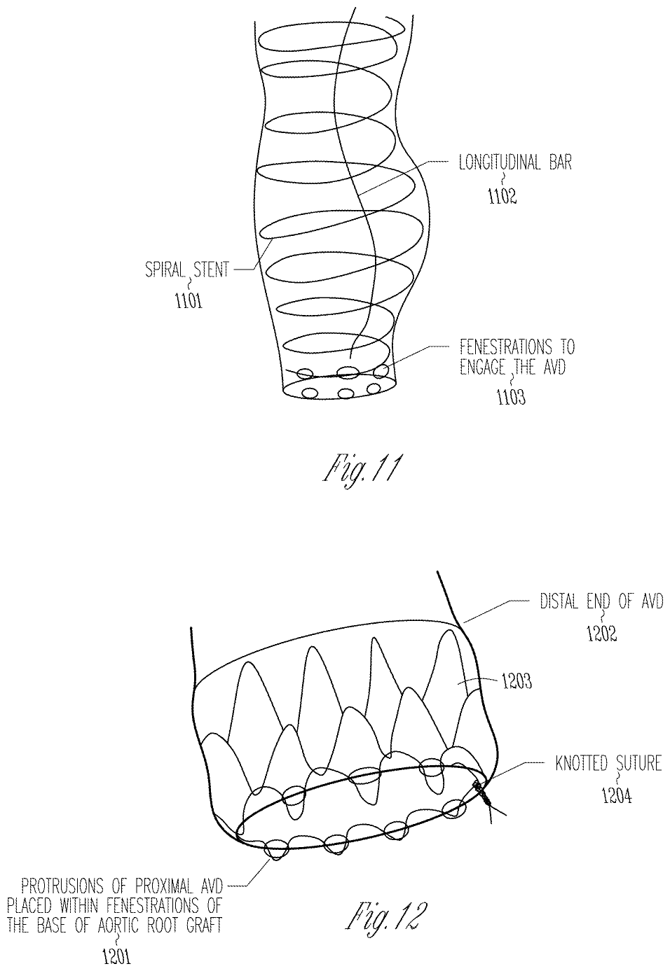

FIG. 11 is a drawing demonstrating an Endo-Bentall device formed as a spiral stent 1101 and a longitudinal bar 1102 and a base with a plurality of fenestrations 1103, according to various embodiments of the present subject matter. In various embodiments, the aortic root graft is formed as a spiral circular stent 1101 starting within or above the “landing zone” of the AVD to increase the radial force and maintain the horizontal space and shape of the aortic root graft up to the distal “landing zone” 816 of the ascending stent graft 101, as well as a longitudinal bar 1102 to support the length and the height of the ascending graft 101. In various embodiments, the stent material anywhere within current embodiments includes nitinol for the components of the Endo-Bentall device, such as the longitudinal bar, spiral stent, proximal or distal landing zone (for example, distal rim 616). In various embodiments, the stent material includes stainless steel for the components of the Endo-Bentall device, such as the longitudinal bar, spiral stent, proximal or distal landing zone (for example, distal rim 616). In various embodiments, the stent material includes nano structures and smart metals with unique characteristics for the components of the Endo-Bentall device, such as the longitudinal bar, spiral stent, proximal or distal landing zone (for example, distal rim 616) or the longitudinal bar.

The Endo-Bentall device example of FIG. 11 is provided to show a plurality of fenestrations in terms of numbers or shapes (i.e. oval, round, slit-like, trapezoid, etc.) to engage an AVD. The base of the graft includes multiple fenestrations to allow for custom valves or commercially available transcatheter valves (such as the Evolute, Engager, or Jena valve types, which have protruding bars at the proximal end of the AVD) to engage in the holes. In various embodiments, connections can be made using one or more of: two or more manually placed non-absorbable sutures on the operating table (attaching the AVD to the base of the Endo-Bentall device permanently), snared down and knotted “skirt” at the base of the Endo-Bentall device containing the AVD (drawn in 318 or 1204). 2-10 round connectors or hooks, magnets, and/or simply using commercially available glue to attach the AVD inside and at the proximal portion of the Endo-Bentall device. In various embodiments, aforementioned connections, may be used for custom valves or commercially available transcatheter valves (such as Sapien 3, Directflow, or Lotus valves, which have smooth or non-protruding design at the proximal end of the AVD). Therefore, many connection options are provided by the present subject matter according to the type of AVD used in the Endo-Bentall device. Thus, the present apparatus can be used with various types of specialized and commercially available, off-the-shelf AVDs.

FIG. 12 is a drawing demonstrating detail of the fenestrations and connections with an AVD 1203 according to various embodiments of the present subject matter. AVD 1203 includes a proximal AVD end 1201 and a distal AVD end 1202. In various embodiments, protrusions of a proximal AVD end 1201 can be inserted into the fenestrations of the base of the Endo-Bentall device to provide a single piece design. The protrusions can be engaged and enhanced using a knotted suture 1204 or any other method of connection.

The present subject matter allows for construction of a single device to repair the aortic valve, aortic root, and ascending aorta. The construction of a one-piece design avoids leakage of blood (similar to type III endoleak in aortic stent grafts) as may be found using approaches with multiple piece of aortic stent grafts. The present design maintains a generous diameter of the base of the aortic root graft portion to avoid an hourglass shape which can generate a pressure gradient by narrowing of the outflow tract in the proximal aorta. Constriction of the lumen, as described in other patents, can result in unnecessarily high pressures in the aorta proximal to the constriction and within the heart and creates a prosthesis-patient mismatch. The present approach avoids this problem and allows for a single piece construction of the Endo-Bentall device with un-obstructed flow of the blood from the left ventricle to the aortic arch.

Examples of Endovascular Delivery of the Device

After proper sizing of the aortic annulus and the distal ascending/aortic arch based on CT imaging, the proper-sized device is ordered based on subject's individual anatomic sizing. The device may arrive as pre-assembled with the specific AVD or may be connected by the surgeon to the first portion of the device using aforementioned approaches and subsequently cinched down into the device delivery system 406 in the operating room.

The patient is anticoagulated with intravenous heparin or similar anti-coagulant medications. A guidewire is placed from femoral artery into the left ventricle. The device is introduced via femoral artery or one of the alternative access routes mentioned above using standard Seldinger technique. Under fluoroscopic and transesophageal guidance the AVD portion of the device 506 is implanted at the aortic valve annulus (under rapid pacing, as needed). The proximal deployment is modified as per instruction for use for individual AVD used in the procedure. The AVD has specific radiopaque markers to allow the surgeon to deploy the device proximally at the intended site. At this time, the heart is pumping the blood through the AVD. Next, the aortic root graft with fenestrations/branched stent grafts is promptly unsheathed from the delivery system 502. Last but not least, the distal ascending aortic or the aortic arch portion is deployed, completing the implantation of the Endo-Bentall device. Next, the delivery system is removed keeping the guide-wire in the left ventricle of the heart, and the access sheath in the patient's femoral or alternative access artery. During this time there is adequate coronary artery perfusion through the fenestrations or branched stent grafts. At this time, further balloon dilatation of the AVD and angioplasty at the distal aortic stent graft landing zone is performed, as needed. Next, coronary artery wires are introduced through the femoral arteries or one of the other access sites into the aorta. These wires would cannulate the fenestrations or the internal conduit and branch stent grafts (depending on the used embodiment). The wires are advanced into the left and right coronary artery system. Next, commercially available coronary stent grafts (are used to connect the fenestration or the proximal end of the branched graft to the coronary ostia of right and left coronary artery. Coronary angiogram confirms patency of the coronary stent grafts, and lack of endoleak, and echocardiogram would confirm the proper function of the valve. Left ventricular angiogram would confirm the proper seal of the AVD and distal stent graft without evidence of endoleak. Any endoleak in the aorta or the coronary stent grafts would prompt balloon angioplasty or additional stent grafting in the desired location. Alternatively, in patients where the aortic root is normal in size, such as type A dissection or ascending aneurysm without aortic root involvement, a coronary stent graft (704) would not be needed as the radial force of the aortic root grafts (314) would allow for apposition of the coronary fenestrations (214) to native left and right coronary arteries.

### EXAMPLES

These examples are intended to demonstrate the present subject matter but are not intended to be an exhaustive or exclusive list of variations, and as such are not offered in a limiting sense.

The present subject matter provides an apparatus for endovascular replacement of an aortic valve, an aortic root, and an ascending aorta of an organism having a heart and coronary arteries. The apparatus may include an assembly including an ascending stent graft, an aortic root graft connected to the ascending stent graft, and an aortic valve device (AVD) connected to the aortic root graft. The ascending stent graft, aortic root graft, and AVD form a unitary device configured for endovascular delivery over a wire and to be in a collapsed state allowing for the endovascular delivery. The ascending stent graft and the aortic root graft are configured to be delivered to the ascending aorta. The AVD is configured to be delivered to the aortic root, proximal the ascending stent graft. The aortic root graft is configured to be connected to the coronary arteries. This assembly may be referred to as an assembled Endo-Bentall device and AVD apparatus, and is a single-piece apparatus for coverage of the aortic root and the ascending aorta. The single-piece configuration reduces the chance of endoleak associated with use of multiple segments to build the aortic coverage. The assembly can include radial and/or longitudinal bars/stents to help with proper apposition in the aortic root and the ascending aorta. This assembly can work with off-the-shelf and FDA-approved transcatheter valve devices, thereby reducing the complexity of having pre-assembled AVD with the Endo-Bentall device. The proximal AVD may be balloon-expandable or self-expanding. In various embodiments, the apparatus includes wherein the AVD is connected to the aortic root graft using a plurality of hooks, a proximal snare and surgical knot, or surgical suturing. In various embodiments, the apparatus includes wherein the AVD includes a proximal portion having protrusions, the aortic root graft includes a base having fenestrations, and the AVD is connected to the aortic root graft by inserting the protrusions into the fenestrations. The fenestrations may be factory-made or hand-made. The protrusions may be engaged and enhanced using a knotted suture or any other method of connection. In various embodiments, the apparatus includes wherein the AVD is connected to the aortic root graft using one or more of sliding or binding surfaces or using one or more of magnetic technology, chemical technology, or nanotechnology. In various embodiments, the apparatus includes wherein the aortic root graft includes an exterior portion configured to be apposed to aortic valve annulus of the organism, the exterior portion made of one or more materials selected to reduce paravalvular leaks. Examples of the one or more materials include foam, gel, other compressible material, or a combination of any of these materials. In some embodiments, the paravalvular leaks can be eliminated. In various embodiments, the apparatus includes wherein the AVD includes a radially stretchable polyester or pTFE for expansion of the AVD to aortic valve annulus of the organism during a cardiac cycle. In various embodiments, the aortic root graft includes the exterior portion configured to be apposed to the aortic valve annulus, and/or the AVD includes the radially stretchable polyester or pTFE for expansion of the AVD to the aortic valve annulus. Use of other materials are possible without departing from the scope of the present subject matter. In various embodiments, the apparatus includes wherein the assembly is sized to prevent narrowing or hourglass shaping of a lumen of the aortic root or the ascending aorta. Such narrowing or hourglass shaping of the inner lumen of the aortic root or the ascending aorta would cause gradients and pressure difference within the assembly and increase afterload and workload for the heart. In various embodiments, the assembly is sized and shaped to be introduced transfemorally or through the axillary, subclavian, carotid artery, or the apex of the heart. The assembly is assembled to be placed in the delivery system in the operation room prior to implantation. In various embodiments, the apparatus includes wherein the aortic root graft includes one or more coronary fenestrations configured for connection to the coronary arteries. In various embodiments, the apparatus includes wherein the aortic root graft includes one or more stent grafted fenestrations configured for connection to the coronary arteries. In various embodiments, the apparatus includes wherein the aortic root graft includes one or more stent graft branches configured for connection to the coronary arteries, and the aortic root graft may be configured to be connected to the coronary arteries through one or more conduits connected to the one or more stent graft branches. In various embodiments, the passageways for connection to the coronary arteries can include one or more internal, partly internal, or external conduits connected to right and left stent graft branches configured to be connected to the right and left coronary arteries, respectively. The internal orifice of each conduit may have an oversized oval or round orifice to facilitate distant coronary wire cannulation. The length of each conduit may vary from starting at the distal portion of the ascending aortic graft to just above the aortic root graft. In various embodiments, the apparatus includes wherein the aortic root graft includes one or more coronary fenestrations, stent grafted fenestrations, or stent graft branches configured for connection to the coronary arteries and positioned on the assembly according to coronary anatomy constellation of the organism. The location of each of the coronary fenestration(s), stent grafted fenestration(s), or stent graft branch(es) is adjusted according to the patient's coronary anatomy constellation. The spatial distance/angulation for patients with aberrant coronary anatomy dictates the location/constellation of fenestrations/coronary stent grafts. The adjustment can be custom-made in the factory, or on the operating table and prior to implantation using a cutting or burning device to produce the proper fenestration locations. In various embodiments, the apparatus includes passageways for connection to the coronary arteries by one or more internal, partly internal, or external conduits connected to right and left stent graft branches configured to be connected to the right and left coronary arteries, respectively, wherein the length of each conduit may vary from starting at a distal portion of the ascending aortic graft to just above the aortic root graft and wherein an internal orifice of each conduit may have an oversized oval or round orifice to facilitate distant coronary wire cannulation.

The present subject matter provides a method for endovascular replacement of an aortic valve, aortic root, and ascending aorta of an organism having a heart and coronary arteries. The method may include delivering an assembly over a wire. The assembly is a unitary device including an ascending stent graft, an aortic root graft connected to the ascending stent graft, and an aortic valve device (AVD) connected to the aortic root graft and in a collapsed state during the delivery. The delivery may include positioning the ascending stent graft and the aortic root graft in the ascending aorta and positioning the AVD in the aortic root, proximal the ascending stent graft. The method may further include connecting the coronary arteries to the aortic root graft. In various embodiments, the method includes delivering the assembly with the AVD connected to the aortic root graft using a plurality of hooks, a proximal snare and surgical knot, or surgical suturing. In various embodiments, the method includes wherein the AVD includes a proximal portion having protrusions, the aortic root graft includes a base having fenestrations, and delivering the assembly comprises delivering the assembly with the AVD connected to the aortic root graft by inserting the protrusions into the fenestrations. In various embodiments, the method includes delivering the assembly with the AVD connected to the aortic root graft using one or more of sliding or binding surfaces or using one or more of magnetic technology, chemical technology, or nanotechnology. In various embodiments, the method includes delivering the assembly sized to prevent narrowing or hourglass shaping of a lumen of the aortic root or the ascending aorta. In various embodiments, the method includes connecting the coronary arteries to the aortic root graft using one or more coronary fenestrations of the aortic root graft. In various embodiments, the method includes connecting the coronary arteries to the aortic root graft using one or more stent grafted fenestrations of the aortic root graft. In various embodiments, the method includes connecting the coronary arteries to the aortic root graft using one or more stent graft branches of the aortic root graft. In various embodiments, the method includes wherein the aortic root graft includes one or more coronary fenestrations, stent grafted fenestrations, or stent graft branches configured for connection to the coronary arteries, and further comprising adjusting one or more locations of the one or more coronary fenestrations, stent grafted fenestrations, or stent graft branches on the assembly according to coronary anatomy constellation of the organism. In various embodiments, the method includes placing the assembly in a delivery system and introducing the assembly transfemorally or through an axillary artery of the organism, a subclavian artery of the organism, a carotid artery of the organism, or an apex of the heart using the delivery system.

The present subject matter provides method (such as Endo-Bentall procedures) and apparatus (such as Endo-Bentall devices) for endovascular replacement of an aortic valve, aortic root, and ascending aorta of an organism having a heart, comprising: delivering an ascending stent graft and aortic root graft in a collapsed state to allow for endovascular delivery, the delivering over a wire to the ascending aorta; delivering an AVD to the aortic root, proximal the ascending stent graft; connecting the AVD to the aortic root graft; and connecting the coronary arteries to the aortic root graft. In various embodiments the method and apparatus includes wherein the AVD is connected to the aortic root graft using a plurality of hooks. In various embodiments the method and apparatus includes wherein the AVD is connected to the aortic root graft using a snare and surgical knot. In various embodiments the method and apparatus includes wherein the AVD is connected to the aortic root graft using suturing. In various embodiments the method and apparatus includes wherein the AVD is connected to the aortic root graft using sliding and/or binding surfaces. In various embodiments the method and apparatus includes wherein the AVD is connected to the aortic root graft using magnetic, chemical, and/or nanotechnology. In various embodiments the method and apparatus includes wherein the aortic root graft includes coronary fenestrations for connection to the coronary arteries. In various embodiments the method and apparatus includes wherein the aortic root graft includes various internal, external or a combination of two conduits for connection to the coronary arteries. In various embodiments the method and apparatus includes wherein the aortic root graft and the ascending stent graft includes stent graft branches for connection to the coronary arteries.

The present subject matter provides method and apparatus for endovascular replacement of an aortic valve, aortic root, and ascending aorta of an organism having a heart, comprising: delivering an assembly comprising an ascending stent graft, an aortic root graft, and an aortic valve device in a collapsed state to allow for endovascular delivery to the heart; and connecting the coronary arteries to the aortic root graft. In various embodiments, the delivery is over the wire. In various embodiments the method and apparatus includes wherein the AVD is connected to the aortic root graft using a plurality of hooks. In various embodiments the method and apparatus includes wherein the AVD is connected to the aortic root graft using a snare and surgical knot. In various embodiments the method and apparatus includes wherein the AVD is connected to the aortic root graft using suturing. In various embodiments the method and apparatus includes wherein the AVD is connected to the aortic root graft using sliding and/or binding surfaces. In various embodiments the method and apparatus includes wherein the AVD is connected to the aortic root graft using magnetic, chemical, and/or nanotechnology. In various embodiments the method and apparatus includes wherein the aortic root graft includes coronary fenestrations for connection to the coronary arteries. In various embodiments the method and apparatus includes wherein the aortic root graft includes various internal, external or a combination of two conduits for connection to the coronary arteries. In various embodiments the method and apparatus includes wherein the aortic root graft and the ascending stent graft includes stent graft branches for connection to the coronary arteries.

Other methods and apparatus would be apparent to a person of skill in the art upon reading and understanding the present subject matter.

This application is intended to cover adaptations or variations of the present subject matter. It is to be understood that the above description is intended to be illustrative, and not restrictive. The scope of the present subject matter should be determined with reference to the appended claims, along with the full scope of legal equivalents to which such claims are entitled.

What is claimed is:

1. An apparatus for endovascular replacement of an aortic valve, an aortic root, and an ascending aorta of an organism having a heart and coronary arteries, comprising:
an assembly including an ascending stent graft, an aortic root graft connected to the ascending stent graft, and an aortic valve device (AVD) connected to the aortic root graft, the ascending stent graft, aortic root graft, and AVD forming a unitary device configured for endovascular delivery over a wire and to be in a collapsed state allowing for the endovascular delivery, the ascending stent graft and the aortic root graft configured to be delivered to the ascending aorta, the AVD configured to be delivered to the aortic root, proximal the ascending stent graft, the aortic root graft configured to be connected to the coronary arteries.

2. The apparatus of claim 1, wherein the AVD is connected to the aortic root graft using a plurality of hooks, a proximal snare and surgical knot, or surgical suturing.

3. The apparatus of claim 1, wherein the AVD includes a proximal portion having protrusions, the aortic root graft includes a base having fenestrations, and the AVD is connected to the aortic root graft by inserting the protrusions into the fenestrations.

4. The apparatus of claim 1, wherein the AVD is connected to the aortic root graft using one or more of sliding or binding surfaces or using one or more of magnetic technology, chemical technology, or nanotechnology.

5. The apparatus of claim 1, wherein the aortic root graft includes an exterior portion configured to be apposed to aortic valve annulus of the organism, the exterior portion made of one or more materials selected to reduce paravalvular leaks.

6. The apparatus of claim 1, wherein the AVD includes a radially stretchable polyester or pTFE for expansion of the AVD to aortic valve annulus of the organism during a cardiac cycle.

7. The apparatus of claim 1, wherein the assembly is sized to prevent narrowing or hourglass shaping of a lumen of the aortic root or the ascending aorta.

8. The apparatus of claim 1, wherein the aortic root graft includes one or more coronary fenestrations configured for connection to the coronary arteries.

9. The apparatus of claim 1, wherein the aortic root graft includes one or more stent grafted fenestrations configured for connection to the coronary arteries.

10. The apparatus of claim 1, wherein the aortic root graft includes one or more stent graft branches configured for connection to the coronary arteries.

11. The apparatus of claim 10, wherein the aortic root graft is configured to be connected to the coronary arteries through one or more conduits connected to the one or more stent graft branches.

12. The apparatus of claim 1, wherein the aortic root graft includes one or more coronary fenestrations, stent grafted fenestrations, or stent graft branches configured for connection to the coronary arteries and positioned on the assembly according to coronary anatomy constellation of the organism.

13. The apparatus of claim 1, including passageways for connection to the coronary arteries by one or more internal, partly internal, or external conduits connected to right and left stent graft branches configured to be connected to the right and left coronary arteries, respectively, wherein the length of each conduit may vary from starting at a distal portion of the ascending aortic graft to just above the aortic root graft and wherein an internal orifice of each conduit may have an oversized oval or round orifice to facilitate distant coronary wire cannulation.

14. A method for endovascular replacement of an aortic valve, aortic root, and ascending aorta of an organism having a heart and coronary arteries, comprising:
delivering an assembly over a wire, the assembly being a unitary device including an ascending stent graft, an aortic root graft connected to the ascending stent graft, and an aortic valve device (AVD) connected to the aortic root graft and in a collapsed state during the delivery, the delivery including positioning the ascending stent graft and the aortic root graft in the ascending aorta and positioning the AVD in the aortic root, proximal the ascending stent graft; and
connecting the coronary arteries to the aortic root graft.

15. The method of claim 14, wherein delivering the assembly comprises delivering the assembly with the AVD connected to the aortic root graft using a plurality of hooks, a proximal snare and surgical knot, or surgical suturing.

16. The method of claim 14, wherein the AVD includes a proximal portion having protrusions, the aortic root graft includes a base having fenestrations, and delivering the assembly comprises delivering the assembly with the AVD connected to the aortic root graft by inserting the protrusions into the fenestrations.

17. The method of claim 14, wherein delivering the assembly comprises delivering the assembly with the AVD connected to the aortic root graft using one or more of sliding or binding surfaces or using one or more of magnetic technology, chemical technology, or nanotechnology.

18. The method of claim 14, wherein delivering the assembly comprises delivering the assembly sized to prevent narrowing or hourglass shaping of a lumen of the aortic root or the ascending aorta.

19. The method of claim 14, wherein connecting the coronary arteries to the aortic root graft comprises connecting the coronary arteries to the aortic root graft using one or more coronary fenestrations of the aortic root graft.

20. The method of claim 14, wherein connecting the coronary arteries to the aortic root graft comprises connecting the coronary arteries to the aortic root graft using one or more stent grafted fenestrations of the aortic root graft.

21. The method of claim 14, wherein connecting the coronary arteries to the aortic root graft comprises connecting the coronary arteries to the aortic root graft using one or more stent graft branches of the aortic root graft.

22. The method of claim 14, wherein the aortic root graft includes one or more coronary fenestrations, stent grafted fenestrations, or stent graft branches configured for connection to the coronary arteries, and further comprising adjusting one or more locations of the one or more coronary fenestrations, stent grafted fenestrations, or stent graft branches on the assembly according to coronary anatomy constellation of the organism.

23. The method of claim 14, wherein delivering the assembly comprises placing the assembly in a delivery system and introducing the assembly transfemorally or through an axillary artery of the organism, a subclavian artery of the organism, a carotid artery of the organism, or an apex of the heart using the delivery system.
